(19) United States
(12) Patent Application Publication
(10) Pub. No.: US 2026/0217733 A1
(43) Pub. Date: Jul. 30, 2026

(54) PROCESS FOR PREPARATION OF RISDIPLAM, NOVEL INTERMEDIATES, AND PROCESS FOR PREPARATION THEREOF

(71) Applicant: HARMAN FINOCHEM LIMITED, Mumbai, Maharashtra (IN)

(72) Inventors: Harpreet Singh MINHAS, Mumbai, Maharashtra (IN); Gurpreeet Singh MINHAS, Mumbai, Maharashtra (IN); Nitin Shardchandra PRADHAN, Thane (West), Maharashtra (IN); Vihar Raghunath TELANGE, Thane, Maharashtra (IN); Akshay Sambhaji ARABUNE, Sangli, Maharashtra (IN); Vaibhav Vasant MARDHEKAR, Thane, Maharashtra (IN); Yuvaraj Suresh SONAR, Thane, Maharashtra (IN)

(21) Appl. No.: 19/144,168

(22) PCT Filed: Dec. 5, 2023
(86) PCT No.: PCT/IN2023/051132

§ 371 Date: Jun. 27, 2025

(30) Foreign Application Priority Data
Jan. 21, 2023 (IN) 202321004252

(51) Int. Cl. C07D 519/00

(52) U.S. Cl. CPC C07D 519/00

(57) ABSTRACT

The present invention discloses processes for the preparation of 7-(4,7-Diazaspiro[2,5] octan-7-yl)-2-(2,8-dimethylimidazo[1,2-b]pyridazin-6-yl)-pyrido[1,2-a]pyrimidin-4-one i.e. Risdiplam of Formula-1 via novel intermediates namely 7-Benzyl-1-formyl-4,7-Diazaspiro[2,5]octane (Formula-2), 1-Formyl-4,7-Diazaspiro[2,5]octane (Formula-3) and 7-(4-formyl-4, 7-diazaspiro[2,5]octan-7-yl)-2-(2,8-dimethylimidazo{1,2-b]pyridazin-6-yl)-4H-pyrido[1,2-a]pyrimidin-4-one (Formula-4), alkyl/aryl-7-Benzyl-4,7-Diazaspiro[2,5]octane-1-carboxylate (Formula-5), alkyl/aryl-4,7-Diazaspiro[2,5]octane-1-carboxylate (Formula-6) and 7-(4-alkoxycarbonyl)-4,7-diazaspiro[2,5]octan-7-yl)-2-(2,8-dimethylimidazo[1,2-b]pyridazin-6-yl)-4H-pyrido[1,2-a]pyrimidin-4-one (Formula-7) as depicted below.

### FILED OF THE INVENTION

The present invention relates to the process for the preparation of 7-(4,7-Diazaspiro[2,5]octan-7-yl)-2-(2,8-dimethylimidazo[1,2-b]pyridazin-6-yl)-pyrido[1,2-a]pyrimidin-4-one i.e. Risdiplam of Formula-1 via its novel intermediates namely 7-Benzyl-1-formyl-4,7-diazaspiro[2,5]octane (Formula-2), 1-formyl-4,7-Diazaspiro[2,5]octane (Formula-3) and 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4), alkyl/aryl-7-Benzyl-4,7-Diazaspiro[2,5]octane-1-carboxylate (Formula-5), alkyl/aryl-4,7-Diazaspiro[2,5]octane-1-carboxylate (Formula-6) and 7-(4-alkoxycarbonyl)-4,7-diazaspiro[2,5]octan-7-yl)-2-(2,8-dimethylimidazo[1,2-b]pyridazin-6-yl)-4H-pyrido[1,2-a]pyrimidin-4-one (Formula-7) as depicted below:

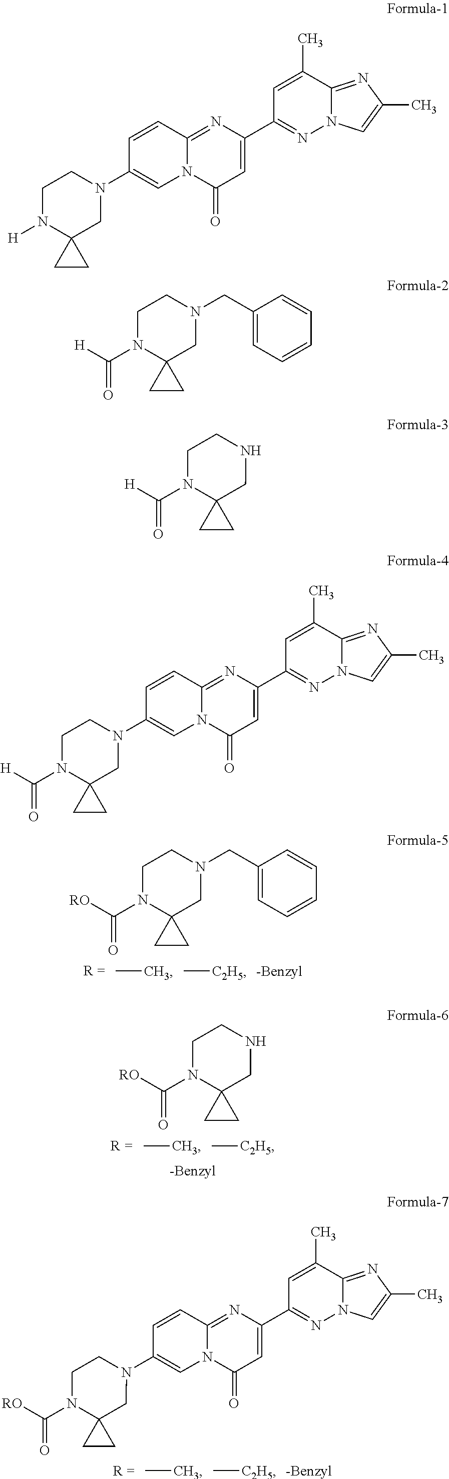

### BACKGROUND OF THE INVENTION

7-(4,7-Diazaspiro[2,5]octan-7-yl)-2-(2,8-dimethylimidazo[1,2-b]pyridazin-6-yl)-pyrido[1,2-a]pyrimidin-4-one (Formula-1) is commonly known as Risdiplam. Risdiplam is a medication used to treat spinal muscular atrophy (SMA) and the first oral medication approved to treat this disease. Risdiplam is approved by the US Food and Drug Administration (FDA) and by European Medicines Agency under the brand name of Evrysdi for the treatment of SMA in patients of 2 months of age and older.

U.S. Pat. No. 9,969,754B2 discloses Risdiplam and its process for the preparation thereof. However, this process results in Risdiplam very poor yields. The process is shown in below scheme 1. According to this process compound (a) is reacted with compound (b) in presence of Pd(Ph₃P)₄ to obtain 2-(2,8-dimethylimidazo[1,2-b]pyridazin-6-yl)-7-fluoro-pyrido[1,2-a]pyrimidin-4-one, which is reacted with 4,7-diazaspiro[2.5]octane hydrochloride to yield Risdiplam.

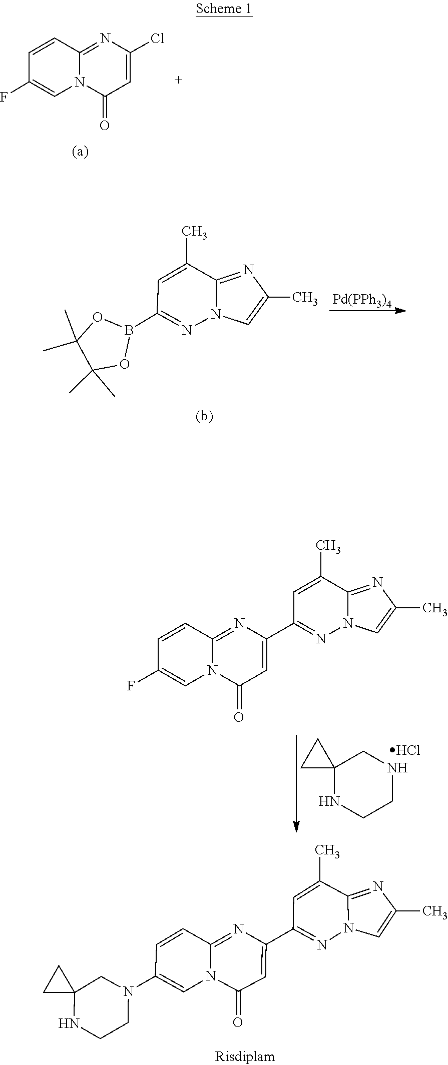

U.S. Pat. No. 11,390,632B2 disclosed the following route of synthesis of Risdiplam, as shown in scheme 2. According to this synthetic scheme, the compound of formula III reacted with compound of formula III′ in the presence of a palladium catalyst or nickel catalyst to form the compound of formula (II); and reacting the compound of formula (II) with a strong acid to obtain a compound of formula (I).

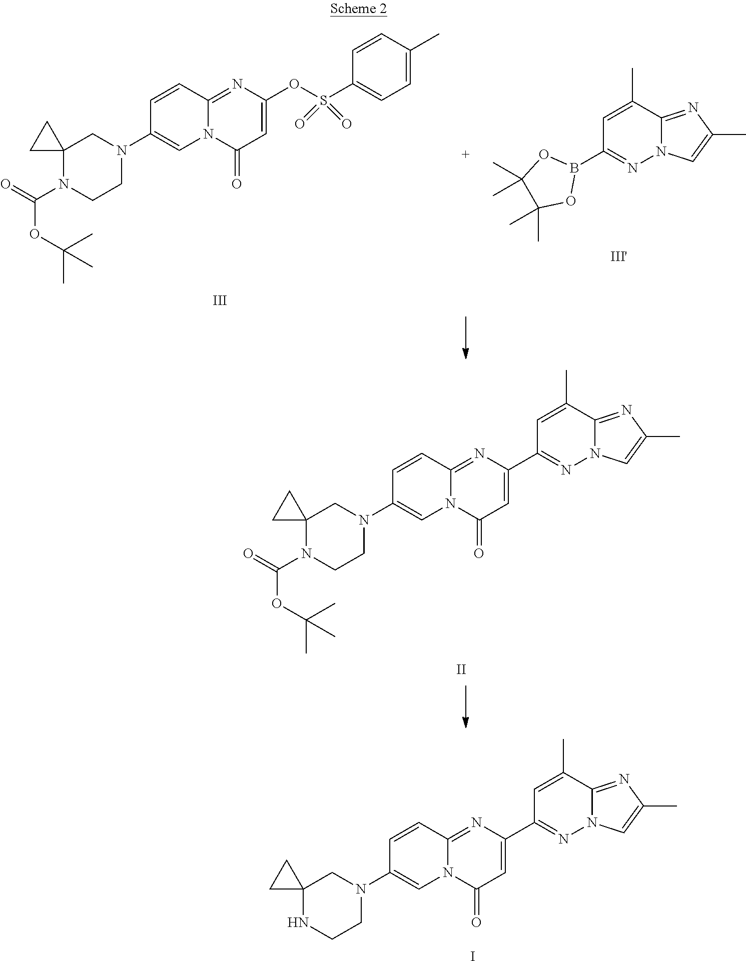

The processes described in the above prior arts resulted in poor yields and they require multiple chromatographic purifications for the isolation of Risdiplam and its intermediates which are considered to be very expensive for commercial scale preparations. Further, WO2020079203 disclosed the crystalline form A of Risdiplam. WO2021021775 and WO2022162107 described crystalline forms of Risdiplam.

Therefore, there remains a need in the art for an improved and industrially applicable process for the preparation of Risdiplam which can overcome the drawbacks of the prior arts processes.

It is therefore an objective of the present invention to provide an efficient process for the preparation of Risdiplam which offer great advantages over the prior art processes in terms of high yield, high purity and less effluents and further simple and suitable for large scale industrial production of Risdiplam.

### SUMMARY OF THE INVENTION

In pursuit of the above objective, the present inventors have developed a novel process for the preparation of Risdiplam that results in the Risdiplam with high purity and yields.

Accordingly, the present invention provides a process for the preparation of Risdiplam compound of formula-1 which process comprises;
a) reacting 7-fluoro-2-(2-methylimidazo[1,2-b]pyridazin-6-yl)-4H-pyrido[1,2-a]pyrimidin-4-one with 1-formyl-4,7-Diazaspiro[2,5]octane (formula-3) in presence of a base and a solvent to obtain 7-(4-Formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4); and

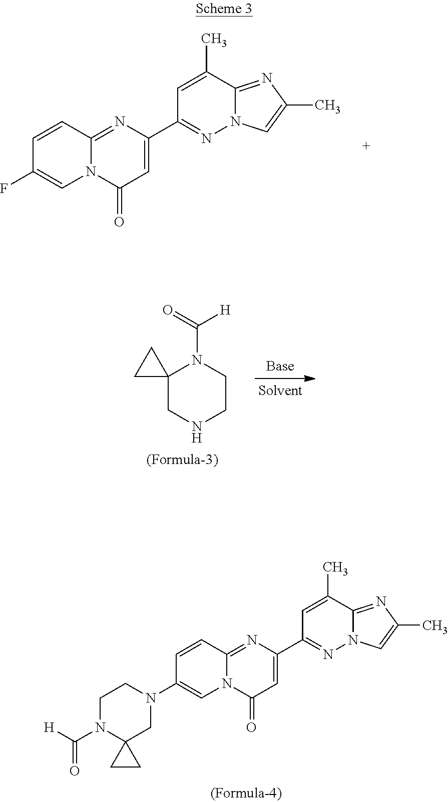 [SMILES: Cc1cn2nc(-c3cc(=O)n4cc(F)ccc4n3)cc(C)c2n1 ; O=CN1CCNCC12CC2 ; Cc1cn2nc(-c3cc(=O)n4cc(N5CCN(C=O)C6(CC6)C5)ccc4n3)cc(C)c2n1]

b) reacting the 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4) with an acid or mixture of acids to obtain 7-(4,7-Diazaspiro[2,5]octan-7-yl)-2-(2,8-dimethylimidazo[1,2-b]pyridazin-6-yl)-pyrido[1,2-a]pyrimidin-4-one i.e. Risdiplam (Formula-1).

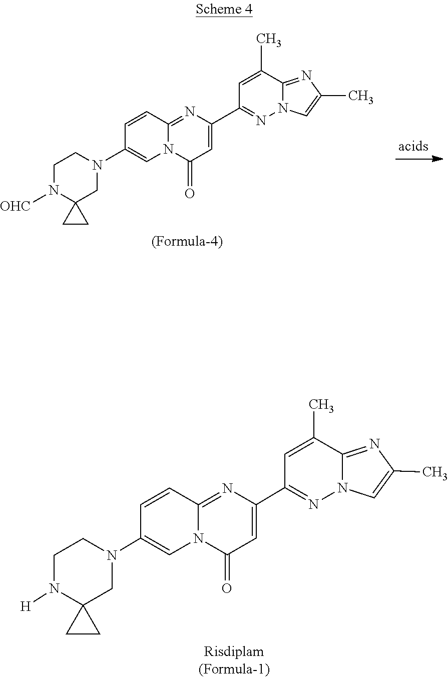 [SMILES: Cc1cn2nc(-c3cc(=O)n4cc(N5CCN(C=O)C6(CC6)C5)ccc4n3)cc(C)c2n1 ; Cc1cn2nc(-c3cc(=O)n4cc(N5CCNC6(CC6)C5)ccc4n3)cc(C)c2n1]

In another preferred aspect, the invention provides a process for preparation of Risdiplam compound of formula-1 which process comprises;
a) Reacting 7-benzyl-4,7-diazaspiro-[2,5]octane with alkyl/aryl chloroformate in presence of a base and solvent to obtain alkyl/aryl-7-Benzyl-4,7-Diazaspiro[2,5]octane-1-carboxylate (formula-5);
b) hydrogenating Alkyl-7-Benzyl-4,7-Diazaspiro[2,5]octane-1-carboxylate (formula-5) in presence of Pd/C in methanol, to obtain alkyl/aryl-4,7-Diazaspiro[2,5]octane-1-carboxylate (formula-6);
c) 7-fluoro-2-(2-methylimidazo[1,2-b]pyridazin-6-yl)-4H-pyrido[1,2-a]pyrimidin-4-one with Alkyl-4,7-Diazaspiro[2,5]octane-1-carboxylate (formula-6) to obtain 7-(4-Alkoxycarbonyl-4,7-diazaspiro[2,5]octan-7-yl)-2-(2,8-dimethylimidazo[1,2-b]pyridazin-6-yl)-4H-pyrido[1,2-a]pyrimidin-4-one (formula-7); and
d) Hydrolyzing 7-(4-Alkoxycarbonyl-4,7-diazaspiro[2,5]octan-7-yl)-2-(2,8-dimethyl imidazo[1,2-b]pyridazin-6-yl)-4H-pyrido[1,2-a]pyrimidin-4-one (Formula-7) in presence of a base/alkali to obtain Risdiplam.
   The process is shown in scheme 5 below.

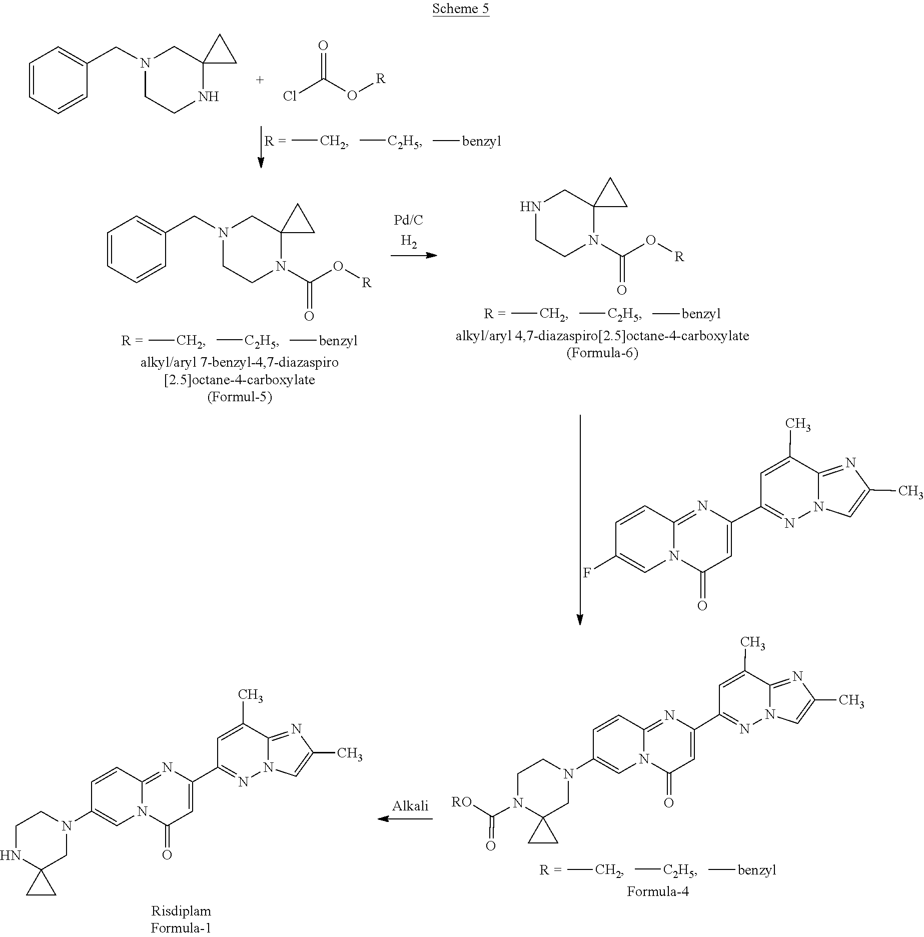

In yet another preferred aspect, the invention provides novel intermediate compounds for preparation of Risdiplam compound of formula-1, selected from the group consisting of; 7-Benzyl-1-formyl-4,7-Diazaspiro[2,5]octane (Formula-2), 1-Formyl-4,7-Diazaspiro[2,5]octane (Formula-3) and 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4), Alkyl/aryl-7-Benzyl-4,7-Diazaspiro[2,5]octane-1-carboxylate (Formula-5), Alkyl/aryl-4,7-Diazaspiro[2,5]octane-1-carboxylate (Formula-6) and 7-(4-alkoxycarbonyl)-4,7-diazaspiro[2,5]octan-7-yl)-2-(2,8-dimethylimidazo[1,2-b]pyridazin-6-yl)-4H-pyrido[1,2-a]pyrimidin-4-one (Formula-7) as depicted below.

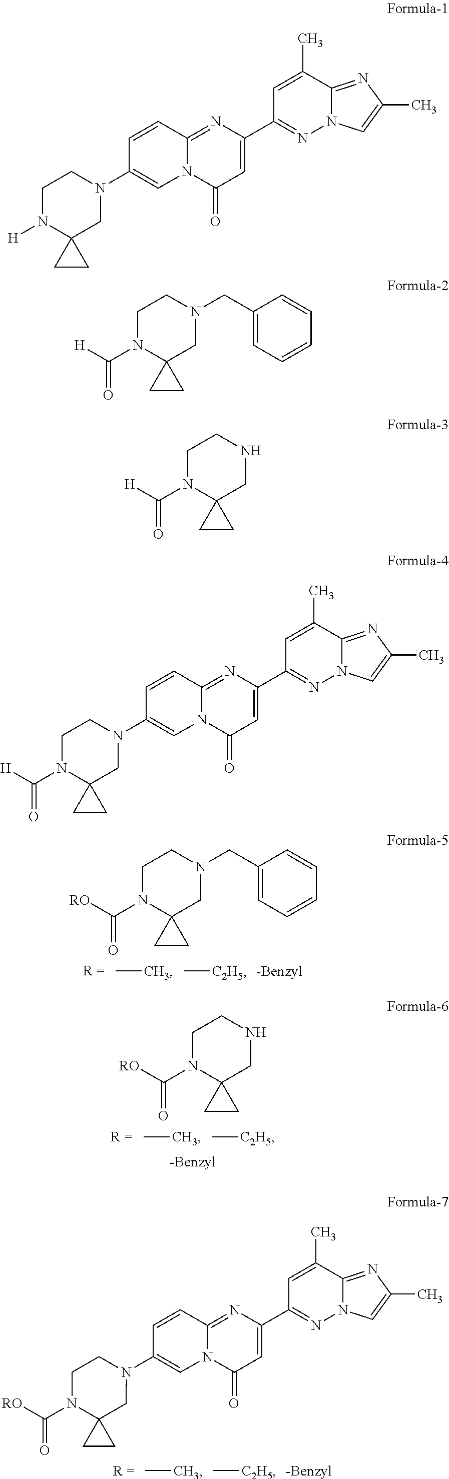

### DETAILED DESCRIPTION OF THE INVENTION

The invention will now be described in detail in connection with certain preferred and optional embodiments, so that various aspects thereof may be more fully understood and appreciated.

In an attempt to develop a novel process for the preparation of Risdiplam and to overcome the disadvantages of prior arts, the present inventors have developed a process which results in high purity and good yield of Risdiplam.

The present invention provides a process for the preparation of 7-(4,7-Diazaspiro[2,5]octan-7-yl)-2-(2,8-dimethylimidazo[1,2-b]pyridazin-6-yl)-pyrido[1,2-a]pyrimidin-4-one i.e. Risdiplam of Formula-1 involving novel intermediates namely 7-Benzyl-1-formyl-4,7-Diazaspiro[2,5]octane (Formula-2), 1-formyl-4,7-Diazaspiro[2,5]octane (Formula-3) and 7-(4-Formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4) as described below:

Accordingly, in an embodiment, the present invention provides a process for the preparation of Risdiplam compound of formula-1 which process comprises;
a) reacting 7-fluoro-2-(2-methylimidazo[1,2-b]pyridazin-6-yl)-4H-pyrido[1,2-a]pyrimidin-4-one with 1-formyl-4,7-Diazaspiro[2,5]octane (formula-3) in presence of a base and solvent to obtain 7-(4-Formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4);

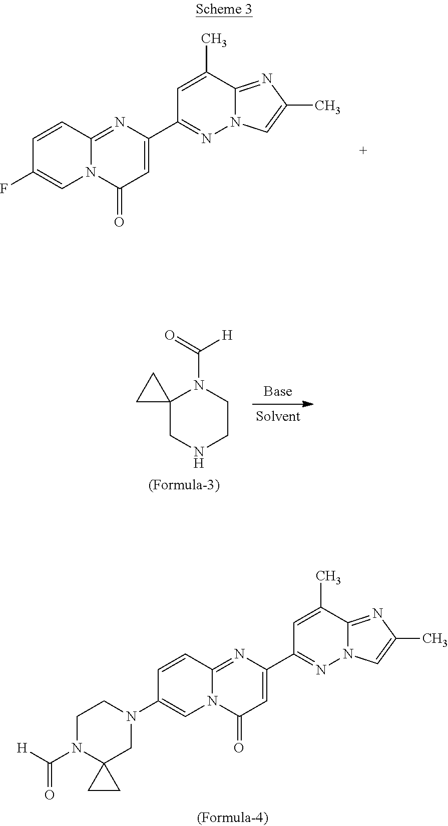 [SMILES: Cc1cn2nc(-c3cc(=O)n4cc(F)ccc4n3)cc(C)c2n1 ; O=CN1CCNCC12CC2 ; Cc1cn2nc(-c3cc(=O)n4cc(N5CCN(C=O)C6(CC6)C5)ccc4n3)cc(C)c2n1]

b) reacting the 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4) with an acid or mixture of acids to obtain 7-(4,7-Diazaspiro[2,5]octan-7-yl)-2-(2,8-dimethylimidazo[1,2-b]pyridazin-6-yl)-pyrido[1,2-a]pyrimidin-4-one i.e. Risdiplam (Formula-1).

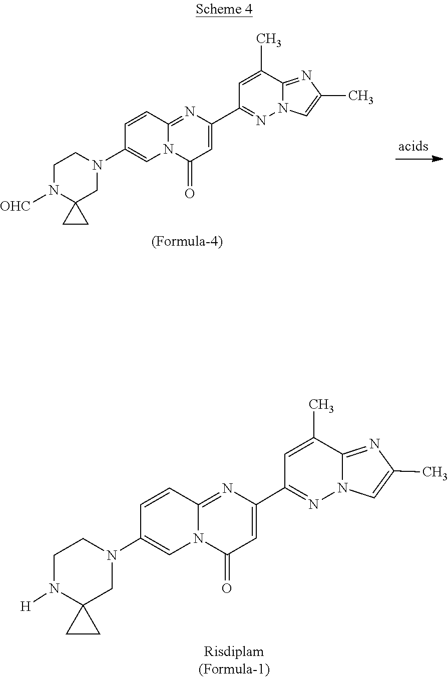 [SMILES: Cc1cn2nc(-c3cc(=O)n4cc(N5CCN(C=O)C6(CC6)C5)ccc4n3)cc(C)c2n1 ; Cc1cn2nc(-c3cc(=O)n4cc(N5CCNC6(CC6)C5)ccc4n3)cc(C)c2n1]

The base used in step (a) is an inorganic base selected from the group consisting of potassium carbonate, sodium carbonate, sodium bicarbonate preferably potassium carbonate.

The solvent used in step (a) is polar aprotic solvents selected from the group consisting of dimethyl formamide (DMF), dimethyl sulfoxide (DMSO) and dimethyl acetamide (DMAc).

The acid(s) used in step (b) is selected from the group consisting of hydrochloric acid, sulphuric acid, acetic acid or combinations thereof.

The present invention further provides a process for the preparation of 1-formyl-4,7-diazaspiro[2,5]octane (formula-3) which process comprises;
a) reacting 7-Benzyl-4,7-diazaspiro[2,5]octane with formic acid in the presence of hydrocarbon solvent to obtain 7-Benzyl-1-formyl-4,7-diazaspiro[2,5]octane (formula-2); and
b) hydrogenating the 7-Benzyl-1-formyl-4,7-diazaspiro[2,5]octane (formula-2) in the presence of a catalyst and an alcoholic solvent to obtain 1-formyl-4,7-diazaspiro[2,5]octane (formula-3).
   The process is shown in scheme 6 below.

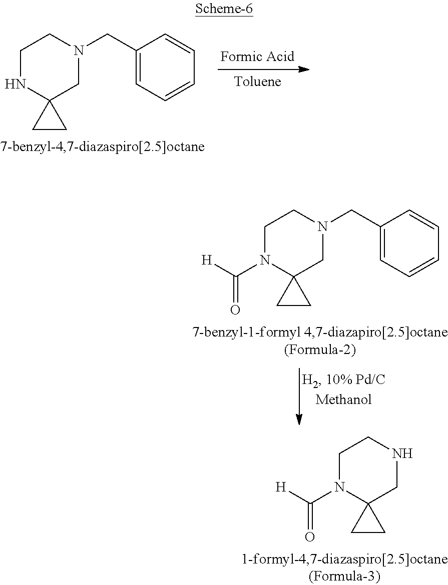 [SMILES: c1ccc(CN2CCNC3(CC3)C2)cc1 ; O=CN1CCN(Cc2ccccc2)CC12CC2 ; O=CN1CCNCC12CC2]

The hydrocarbon solvent used in step (a) is selected from toluene, xylene, halogenated hydrocarbon solvents such as methylene chloride, ethylene chloride etc.

The catalyst in step b) is selected from Raney nickel or Palladium/C, preferably, the catalyst is 10% Pd/C.

The alcoholic solvent used in step b) is a C₁-C₄ alcohol selected from methanol, ethanol, propanol, isopropanol, butanol, isobutanol.

In a further embodiment, the present invention provides a process for the preparation of an intermediate, 7-Benzyl-1-formyl-4,7-diazaspiro[2,5]octane (formula-2) which process comprises; reacting 7-Benzyl-4,7-diazaspiro[2,5]octane with formic acid in the presence of hydrocarbon solvent to obtain 7-Benzyl-1-formyl-4,7-diazaspiro[2,5]octane (formula-2). The process is shown in scheme 7.

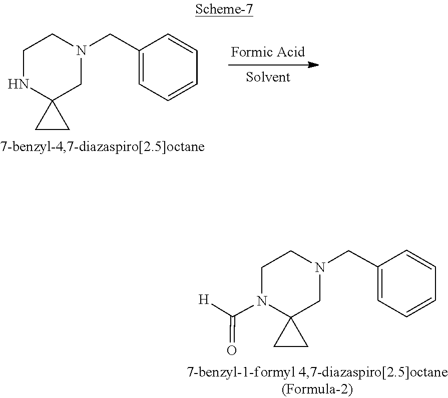 [SMILES: c1ccc(CN2CCNC3(CC3)C2)cc1 ; O=CN1CCN(Cc2ccccc2)CC12CC2]

The hydrocarbon solvent used in the above process is selected from toluene, xylene, ethylene dichloride or methylene dichloride etc.

In yet another embodiment, the invention provides a process for preparation of 7-(4-Formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4), which process comprises; reaction of 7-fluoro-2-(2-methylimidazo[1,2-b]pyridazin-6-yl)-4H-pyrido[1,2-a]pyrimidin-4-one with 1-formyl-4,7-Diazaspiro[2,5]octane (formula-3) in presence of a base and solvent to obtain 7-(4-Formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4).

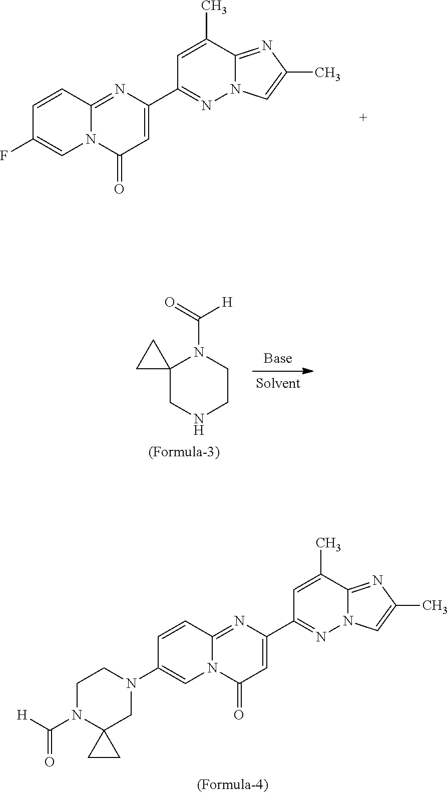 [SMILES: Cc1cn2nc(-c3cc(=O)n4cc(F)ccc4n3)cc(C)c2n1 ; O=CN1CCNCC12CC2 ; Cc1cn2nc(-c3cc(=O)n4cc(N5CCN(C=O)C6(CC6)C5)ccc4n3)cc(C)c2n1]

The base is an inorganic base selected from the group consisting of potassium carbonate, sodium carbonate, sodium bicarbonate, preferably potassium carbonate.

The solvent is a polar aprotic solvent(s) selected from the group consisting of dimethyl formamide (DMF), dimethyl sulfoxide (DMSO) and, dimethyl acetamide (DMAc).

The present invention provides an alternate process for the preparation of 7-(4,7-Diazaspiro[2,5]octan-7-yl)-2-(2,8-dimethylimidazo[1,2-b]pyridazin-6-yl)-pyrido[1,2-a]pyrimidin-4-one i.e. Risdiplam of Formula-1 involving novel intermediates namely alkyl/aryl-7-Benzyl-4,7-Diazaspiro[2,5]octane-1-carboxylate (Formula-5), alkyl/aryl-4,7-Diazaspiro[2,5]octane-1-carboxylate (Formula-6) and 7-(4-Formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4) as described below:

Accordingly, in an alternate embodiment, the invention provides process for preparation of Risdiplam compound of formula-1, which process comprises;
a) Reacting 7-Benzyl-4,7-diazaspiro-[2,5]octane with methyl chloroformate in presence of a base and solvent to obtain Methyl-7-Benzyl-4,7-Diazaspiro[2,5]octane-1-carboxylate (Formula 5);
b) hydrogenating Methyl-7-Benzyl-4,7-Diazaspiro[2,5]octane-1-carboxylate (Formula 5) in presence of Pd/C in methanol, to obtain Methyl-4,7-Diazaspiro[2,5]octane-1-carboxylate (Formula 6);
c) 7-fluoro-2-(2-methylimidazo[1,2-b]pyridazin-6-yl)-4H-pyrido[1,2-a]pyrimidin-4-one with Methyl-4,7-Diazaspiro[2,5]octane-1-carboxylate (Formula 6) to obtain 7-(4-Methoxycarbonyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4); and
d) Hydrolyzing 7-(4-Methoxycarbonyl-4,7-diazaspiro[2,5]octan-7-yl)-2-(2,8-dimethylimidazo{1,2-b]pyridazin-6-yl)-4H-pyrido[1,2-a]pyrimidin-4-one (formula 4) in presence of a base to obtain Risdiplam (Formula-I).

The base and solvent used in step a) is an inorganic base and the solvent is a hydrocarbon solvent. The inorganic base used is selected from the group consisting of potassium carbonate, sodium carbonate, preferably sodium bicarbonate and the hydrocarbon solvent is selected from Toluene and Xylene.

The base used in step c) is selected from the group consisting of potassium carbonate or sodium carbonate, preferably potassium bicarbonate.

The solvent used in step (c) is a polar aprotic solvent selected from the group consisting of dimethyl formamide (DMF), dimethyl sulfoxide (DMSO) and dimethyl acetamide (DMAc).

The base used in step d) is selected from the group consisting of potassium carbonate, sodium carbonate, sodium hydroxide and potassium hydroxide.

In another embodiment, the invention provides process for preparation of Methyl-7-Benzyl-4,7-Diazaspiro[2,5]octane-1-carboxylate of formula 5, which process comprises; Reacting 7-Benzyl-4,7-diazaspiro-[2,5]octane with methyl chloroformate in presence of a base and solvent to obtain Methyl-7-Benzyl-4,7-Diazaspiro[2,5]octane-1-carboxylate of formula 5.

The base and solvent can be selected from an inorganic base and hydrocarbon solvent. The inorganic base is selected from the group consisting of potassium carbonate, sodium carbonate, preferably sodium bicarbonate and the hydrocarbon solvent may be selected from Toluene and Xylene.

In yet another embodiment, the invention provides a process for preparation of Methyl-4,7-Diazaspiro[2,5]octane-1-carboxylate of formula-6, which process comprises hydrogenating Methyl-7-Benzyl-4,7-Diazaspiro[2,5]octane-1-carboxylate of formula 5 in presence of Pd/C in methanol.

In a further embodiment, the present invention provides various novel intermediates that are useful in the synthesis of Risdiplam, as discussed below. Accordingly, in an embodiment, the invention provides 7-Benzyl-1-formyl-4,7-Diazaspiro[2,5]octane, compound of Formula-2, as shown below.

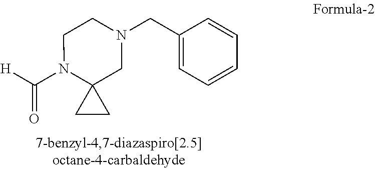 [SMILES: O=CN1CCN(Cc2ccccc2)CC12CC2]

According to another embodiment, the invention provides 1-Formyl-4,7-Diazaspiro[2,5]octane, compound of Formula-3, as shown below.

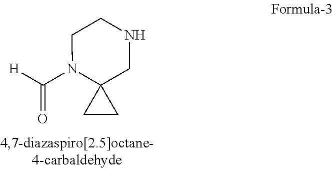

In yet another embodiment, the invention provides 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one, compound of Formula-4, as shown below.

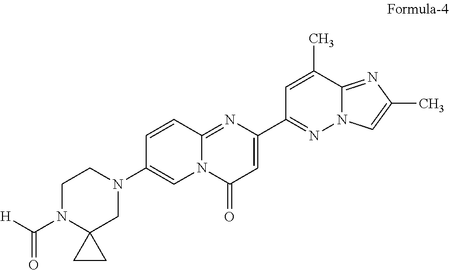 [SMILES: Cc1cn2nc(-c3cc(=O)n4cc(N5CCN(C=O)C6(CC6)C5)ccc4n3)cc(C)c2n1]

In yet another embodiment, the invention provides alkyl/aryl-7-Benzyl-4,7-Diazaspiro[2,5]octane-1-carboxylate, compound of Formula-5, as shown below.

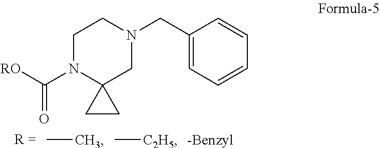

In yet another embodiment, the invention provides alkyl/aryl-4,7-Diazaspiro[2,5]octane-1-carboxylate, compound of Formula-6, as shown below.

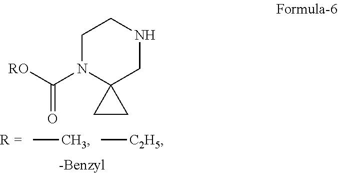

In yet another embodiment, the invention provides 7-(4-alkoxycarbonyl)-4,7-diazaspiro[2,5]octan-7-yl)-2-(2,8-dimethylimidazo[1,2-b]pyridazin-6-yl)-4H-pyrido[1,2-a]pyrimidin-4-one, compound of Formula-7, as shown below.

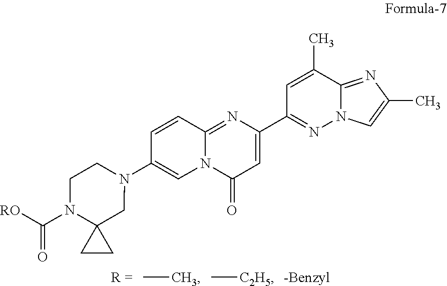

Thus, according to one of the preferred embodiments, the invention discloses a process for synthesis of Risdiplam involving the following novel intermediate compounds selected from the group consisting of; 7-Benzyl-1-formyl-4,7-Diazaspiro[2,5]octane (Formula-2), 1-Formyl-4,7-Diazaspiro[2,5]octane (Formula-3) and 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4), alkyl/aryl-7-Benzyl-4,7-Diazaspiro[2,5]octane-1-carboxylate (Formula-5), alkyl/aryl-4,7-Diazaspiro[2,5]octane-1-carboxylate (Formula-6) and 7-(4-alkoxycarbonyl)-4,7-diazaspiro[2,5]octan-7-yl)-2-(2,8-dimethylimidazo[1,2-b]pyridazin-6-yl)-4H-pyrido[1,2-a]pyrimidin-4-one (Formula-7).

### EXAMPLES

The following examples are illustrative of some of the embodiments of the present invention described herein. These examples should not be considered to limit the spirit or scope of the invention in any way.

### Example-1: Preparation of 7-Benzyl-1-formyl-4,7-Diazaspiro[2.5]octane (Formula-2)

Formic acid (11 g) was added to the mixture of 7-Benzyl-4,7-diazaspiro-[2,5]octane (35 g) and toluene (350 ml) at 30-35° C. The temperature of the reaction mass was raised to reflux at 106-110° C. and removed water azeotropically. After complete removal of water, cooled the reaction mass to 30-35° C. Aqueous sodium carbonate solution (100 ml) was added to the reaction mass and organic layer separated. Distilled off toluene completely from organic layer to get the title compound as oil.

Yield: 35 g (87.83%); LCMS=231 (M+H)

### Example-2: Preparation of 1-formyl-4,7-Diazaspiro[2.5]octane (Formula-3)

A mixture of 7-Benzyl-1-formyl-4,7-Diazaspiro[2,5]octane (100 g), methanol (1000 ml), and 10% Pd/C (20 g) was added in hydrogenator at 30-35° C. and applied hydrogen pressure under stirring for debenzylation. After completion of reaction, reaction mass was filtered and washed with methanol (100 ml) and distilled off organic solvent completely to get the title compound as oil.

Yield: 55 g (90.36%)

### Example-3: Preparation of 7-(4-Formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4)

A mixture of 7-fluoro-2-(2-methylimidazo[1,2-b]pyridazin-6-yl)-4H-pyrido[1,2-a]pyrimidin-4-one (10 g) and 1-formyl-4,7-Diazaspiro[2,5]octane (5 g) were stirred in DMSO (200 ml) with K₂CO₃ (5 μm) at 120-160° C. overnight. The solvent was removed under high vacuum to get crude product which is purified to afford title compound as solid.

Yield: 7 g (50.3%); LCMS=430 (M+H)

### Example-4: Preparation of 7-(4,7-Diazaspiro[2.5]octan-7-yl)-2-(2,8-dimethylimidazo[1,2-b]pyridazin-6-yl)-pyrido[1,2-a]pyrimidin-4-one (Risdiplam)

A mixture of 7-(4-formyl-4,7-diazaspiro[2.5]octan-7-yl)-2-(2,8-dimethylimidazo{1,2-b]pyridazin-6-yl)-4H-pyrido[1,2-a]pyrimidin-4-one (10 g) and acetic acid (50 ml) was added conc. HCl (50 ml). The reaction mass was heated to 80-100° C. till complete deformylation. The reaction mass was cooled to 30-35° C. and 20% aqueous sodium hydroxide (10 ml) was added. The precipitate was filtered off, washed with water and dried under reduced pressure at 60° C. to obtain 7-(4,7-Diazaspiro[2.5]octan-7-yl)-2-(2,8-dimethylimidazo[1,2-b]pyridazin-6-yl)-pyrido[1,2-a]pyrimidin-4-one i.e. Risdiplam as yellow crystals.

Yield: 7.5 g (80.2%); LCMS=402 (M+H)

### Example-5: Preparation of Methyl-7-Benzyl-4,7-Diazaspiro[2.5]octane-1-carboxylate (Formula-5)

Methyl chloroformate (26 g) was added to the mixture of 7-Benzyl-4,7-diazaspiro-[2.5]octane (40 g), sodium carbonate (42 g) and toluene (300 ml) at 30-35° C. The temperature of the reaction mass was raised to reflux at 106-110° C. After complete conversion, cooled the reaction mass to 30-35° C. and added water (160 ml) under stirring. The organic toluene layer was separated, distilled off toluene completely from organic layer to get the title compound as oil.

Yield: 48 g (93.23%); LCMS=261 (M+H)

### Example-6: Preparation of Methyl-4,7-Diazaspiro[2.5]octane-1-carboxylate (Formula-6)

A mixture of Methyl-7-Benzyl-4,7-Diazaspiro[2,5]octane-1-carboxylate (100 g), methanol (1000 ml) and 10% Pd/C (15 g) was added in hydrogenator at 30-35° C. and applied hydrogen pressure under stirring for debenzylation. After completion of reaction, reaction mass is filtered and washed with methanol (100 ml) and distilled off organic solvent completely to get the title compound as oil.

Yield: 55 g (84.11%); LCMS=171 (M+H)

### Example-7: Preparation of 7-(4-Methoxycarbonyl-4,7-diazaspiro[2.5]octan-7-yl)-2-(2,8-dimethylimidazo{1,2-b]pyridazin-6-yl)-4H-pyrido[1,2-a]pyrimidin-4-one (Formula-4)

A mixture of 7-fluoro-2-(2-methylimidazo[1,2-b]pyridazin-6-yl)-4H-pyrido[1,2-a]pyrimidin-4-one (10 g) and Methyl-4,7-Diazaspiro[2,5]octane-1-carboxylate (5.5 g) were stirred in DMSO (200 ml) with K₂CO₃ (5 μm) at 120-160° C. overnight. The solvent was removed under high vacuum to get crude product which was purified to afford title compound as solid. Yield: 6.8 g (45.67%); LCMS=460 (M+H)

### Example-8: Preparation of 7-(4,7-Diazaspiro[2.5]octan-7-yl)-2-(2,8-dimethylimidazo[1,2-b]pyridazin-6-yl)-pyrido[1,2-a]pyrimidin-4-one (Risdiplam)

A mixture of 7-(4-Methoxycarbonyl-4,7-diazaspiro[2,5]octan-7-yl)-2-(2,8-dimethylimidazo[1,2-b]pyridazin-6-yl)-4H-pyrido[1,2-a]pyrimidin-4-one (10 g), sodium hydroxide (2 μm) in water (50 ml) was heated to 95-100° C. till complete conversion. After complete conversion, the reaction mass was cooled to 30-35° C. and filtered off the solid, washed with water and dried under reduced pressure at 60° C. to obtain 7-(4,7-Diazaspiro[2.5]octan-7-yl)-2-(2,8-dimethylimidazo[1,2-b]pyridazin-6-yl)-pyrido[1,2-a]pyrimidin-4-one i.e. Risdiplam as yellow crystals.

Yield: 6.0 g (68.65%); LCMS=402 (M+H)

1. A process for the preparation of Risdiplam compound of Formula-1 which comprises:
a) reacting 7-fluoro-2-(2-methylimidazo[1,2-b]pyridazin-6-yl)-4H-pyrido[1,2-a]pyrimidin-4-one with 1-formyl-4,7-diazaspiro[2,5]octane (formula-3) in the presence of a base and a solvent to obtain 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4) 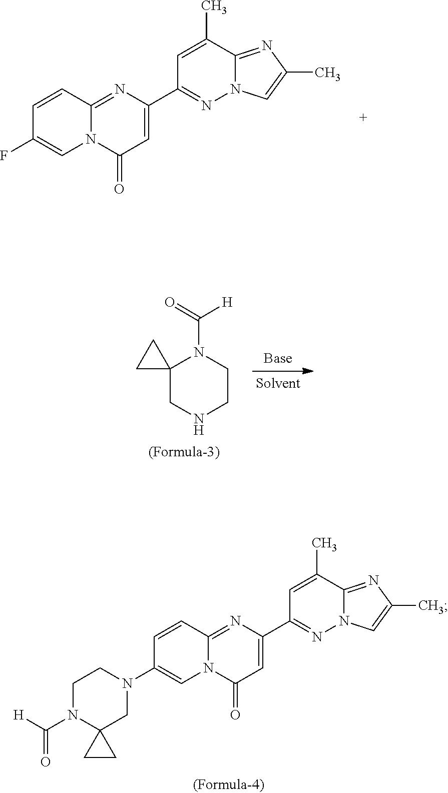 [SMILES: Cc1cn2nc(-c3cc(=O)n4cc(F)ccc4n3)cc(C)c2n1 ; O=CN1CCNCC12CC2 ; Cc1cn2nc(-c3cc(=O)n4cc(N5CCN(C=O)C6(CC6)C5)ccc4n3)cc(C)c2n1]
b) reacting the 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4) with an acid or a mixture of acids to obtain 7-(4,7-diazaspiro[2,5]octan-7-yl)-2-(2,8-dimethylimidazo[1,2-b]pyridazin-6-yl)-pyrido[1,2-a]pyrimidin-4-one (Formula-1)

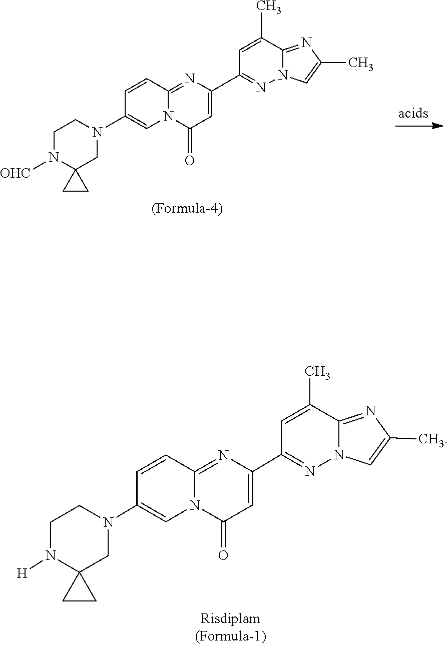 [SMILES: Cc1cn2nc(-c3cc(=O)n4cc(N5CCN(C=O)C6(CC6)C5)ccc4n3)cc(C)c2n1 ; Cc1cn2nc(-c3cc(=O)n4cc(N5CCNC6(CC6)C5)ccc4n3)cc(C)c2n1]

2. The process as claimed in claim 1, wherein, the base used in step a) is an inorganic base selected from the group consisting of potassium carbonate, sodium carbonate, and sodium bicarbonate and the solvent used in step a) is a polar aprotic solvent selected from the group consisting of dimethyl formamide (DMF), dimethyl sulfoxide (DMSO), and dimethyl acetamide (DMAc).

3. (canceled)

4. The process as claimed in claim 1, wherein, the acid or the mixture of acid used in step b) is selected from the group consisting of hydrochloric acid, sulphuric acid, acetic acid, and a combinations thereof.

5. A process for the preparation of 1-formyl-4,7-diazaspiro[2,5]octane (Formula-3) which comprises:
a) reacting 7-benzyl-4,7-diazaspiro[2,5]octane with formic acid in the presence of a hydrocarbon solvent to obtain 7-benzyl-1-formyl-4,7-diazaspiro[2,5]octane (Formula-2); and
b) hydrogenating the 7-benzyl-1-formyl-4,7-diazaspiro[2,5]octane (Formula-2) in the presence of a catalyst and an alcoholic solvent to obtain 1-formyl-4,7-diazaspiro[2,5]octane (Formula-3)

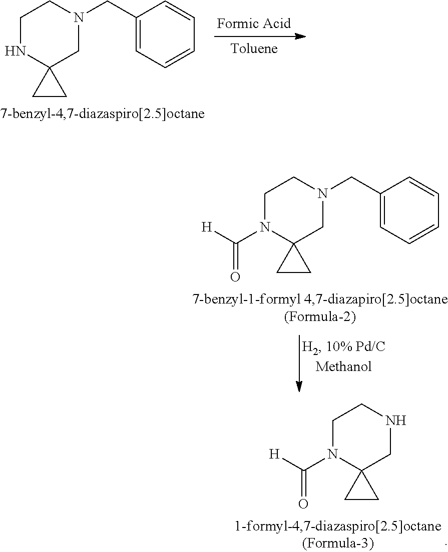 [SMILES: c1ccc(CN2CCNC3(CC3)C2)cc1 ; O=CN1CCN(Cc2ccccc2)CC12CC2 ; O=CN1CCNCC12CC2]

6. The process as claimed in claim 5, wherein the hydrocarbon solvent used in step a) is selected from toluene, xylene, methylene chloride or ethylene chloride.

7. The process as claimed in claim 5, wherein; the alcoholic solvent used in step b) is a C1-C4 alcohol selected from methanol, ethanol, propanol, isopropanol, butanol, isobutanol.

8. A process for the preparation of the intermediate 7-benzyl-1-formyl-4,7-diazaspiro[2,5]octane (Formula-2) as claimed in claim 5 which comprises: reacting 7-benzyl-4,7-diazaspiro[2,5]octane with formic acid in the presence of a hydrocarbon solvent to obtain 7-benzyl-1-formyl-4,7-diazaspiro[2,5]octane (Formula-2)

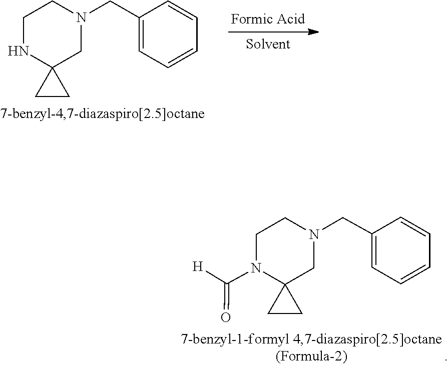 [SMILES: c1ccc(CN2CCNC3(CC3)C2)cc1 ; O=CN1CCN(Cc2ccccc2)CC12CC2]

9. The process as claimed in claim 8, wherein the hydrocarbon solvent is selected from toluene, xylene, methylene chloride, or ethylene chloride.

10. A process for the preparation of 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4) as claimed in claim 1 which comprises: reacting 7-fluoro-2-(2-methylimidazo[1,2-b]pyridazin-6-yl)-4H-pyrido[1,2-a]pyrimidin-4-one with 1-formyl-4,7-diazaspiro[2,5]octane (Formula-3) in the presence of a base and solvent to obtain 7-(4-formyl)-4,7-diazaspiro[2,5]octan-7-yl)-2-(2,8-dimethylimidazo{1,2-b]pyridazin-6-yl)-4H-pyrido[1,2-a]pyrimidin-4-one (Formula-4)

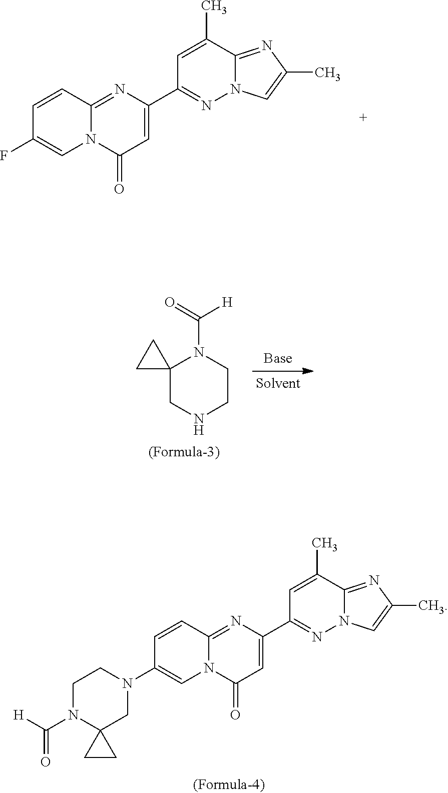 [SMILES: Cc1cn2nc(-c3cc(=O)n4cc(F)ccc4n3)cc(C)c2n1 ; O=CN1CCNCC12CC2 ; Cc1cn2nc(-c3cc(=O)n4cc(N5CCN(C=O)C6(CC6)C5)ccc4n3)cc(C)c2n1]

11. The process as claimed in claim 10, wherein the base is an inorganic base selected from the group consisting of potassium carbonate, sodium carbonate, and sodium bicarbonate and the solvent is a polar aprotic solvent(s) selected from the group consisting of dimethyl formamide (DMF), dimethyl sulfoxide (DMSO), and dimethyl acetamide (DMAc).

12. (canceled)

13. A process for preparation of Risdiplam compound of Formula-1 which comprises:
a) reacting 7-benzyl-4,7-diazaspiro-[2,5]octane with methyl chloroformate in the presence of a base and solvent to obtain methyl-7-benzyl-4,7-diazaspiro[2,5]octane-1-carboxylate (Formula-5);
b) hydrogenating methyl-7-benzyl-4,7-diazaspiro[2,5]octane-1-carboxylate (Formula 5) in the presence of Pd/C in methanol to obtain methyl-4,7-diazaspiro[2,5]octane-1-carboxylate (Formula-6);
c) 7-fluoro-2-(2-methylimidazo[1,2-b]pyridazin-6-yl)-4H-pyrido[1,2-a]pyrimidin-4-one with methyl-4,7-diazaspiro[2,5]octane-1-carboxylate (Formula-6) in a base and in a solvent to obtain 7-(4-methoxycarbonyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4); and
d) hydrolyzing 7-(4-methylcarbonyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4) in the presence of a base to obtain Risdiplam (Formula-1).

14. The process as claimed in claim 13, wherein the base used in step a) is an inorganic base and the solvent used in step a) is a hydrocarbon solvent.

15. The process as claimed in claim 13, wherein the inorganic base is selected from the group consisting of potassium carbonate and sodium carbonate and the hydrocarbon solvent is selected from toluene and xylene.

16. The process as claimed in claim 13, wherein the base used in step c) is selected from the group consisting of potassium carbonate and sodium carbonate and the solvent used in step c) is a polar aprotic solvent selected from the group consisting of dimethyl formamide (DMF), dimethyl sulfoxide (DMSO), and dimethyl acetamide (DMAc).

17. (canceled)

18. The process as claimed in claim 13, wherein the base used in step d) is selected from the group consisting of potassium carbonate, sodium carbonate, sodium hydroxide, and potassium hydroxide.

19. A process for the preparation of methyl-7-benzyl-4,7-diazaspiro[2,5]octane-1-carboxylate of Formula-5 as claimed in claim 13 which comprises reacting 7-benzyl-4,7-diazaspiro-[2,5]octane with methyl chloroformate in the presence of a base and a solvent to obtain methyl-7-benzyl-4,7-diazaspiro[2,5]octane-1-carboxylate of Formula-5.

20. The process for preparation of Risdiplam as claimed in claim 19, wherein the base is an inorganic base selected from the group consisting of potassium carbonate and sodium carbonate and the solvent is a hydrocarbon solvent selected from toluene and xylene.

21. A process for preparation of methyl-4,7-diazaspiro[2,5]octane-1-carboxylate of Formula-6 as claimed in claim 13 which comprises: hydrogenating methyl-7-benzyl-4,7-diazaspiro[2,5]octane-1-carboxylate of Formula-5 in the presence of Pd/C in methanol.

22. Novel intermediate compounds for the synthesis of Risdiplam selected from the group consisting of 7-benzyl-1-formyl-4,7-diazaspiro[2,5]octane (Formula-2), 1-formyl-4,7-diazaspiro[2,5]octane (Formula-3) and 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4), alkyl/aryl-7-benzyl-4,7-diazaspiro[2,5]octane-1-carboxylate (Formula-5), alkyl/aryl-4,7-diazaspiro[2,5]octane-1-carboxylate (Formula-6) and 7-(4-alkoxycarbonyl)-4,7-diazaspiro[2,5]octan-7-yl)-2-(2,8-dimethylimidazo[1,2-b]pyridazin-6-yl)-4H-pyrido[1,2-a]pyrimidin-4-one (Formula-7) as depicted below

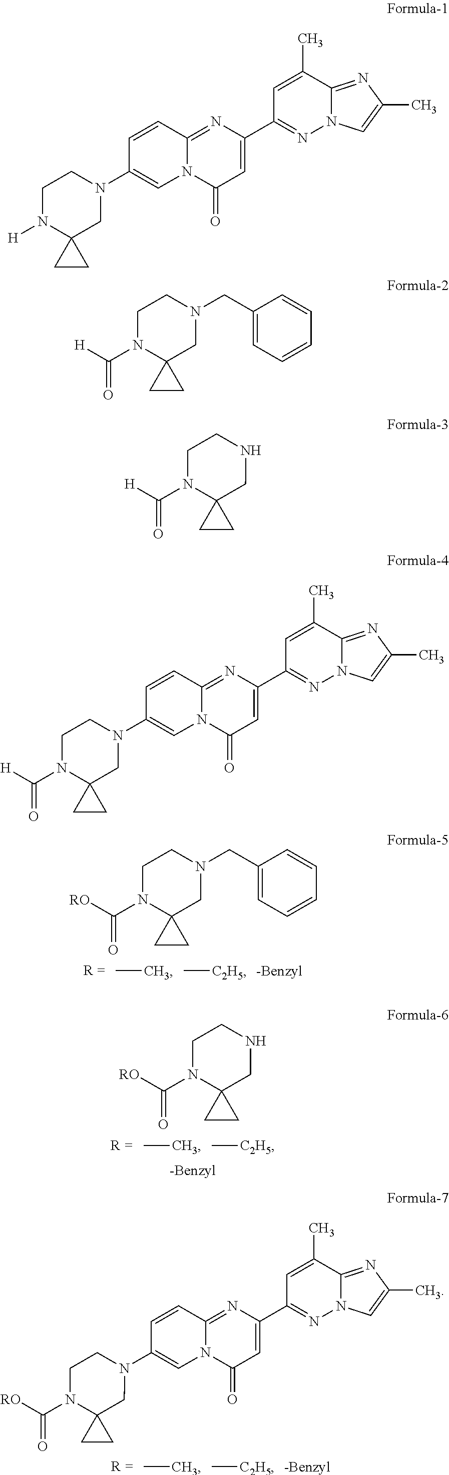

23. A process for the synthesis of Risdiplam involving the following novel intermediate compounds selected from the group consisting of 7-benzyl-1-formyl-4,7-diazaspiro[2,5]octane (Formula-2), 1-formyl-4,7-diazaspiro[2,5]octane (Formula-3) and 7-(4-formyl-4,7-diazaspiro[2,5]octan-7-yl)-2-(2,8-dimethylimidazo{1,2-b]pyridazin-6-yl)-4H-pyrido[1,2-a]pyrimidin-4-one (Formula-4), alkyl/aryl-7-benzyl-4,7-diazaspiro[2,5]octane-1-carboxylate (Formula-5), alkyl/aryl-4,7-diazaspiro[2,5]octane-1-carboxylate (Formula-6) and 7-(4-alkoxycarbonyl)-4,7-diazaspiro[2,5]octan-7-yl)-2-(2,8-dimethylimidazo[1,2-b]pyridazin-6-yl)-4H-pyrido[1,2-a]pyrimidin-4-one (Formula-7) as claimed is claim 22 [chemical structure]
